(19) United States
(12) Patent Application Publication
(10) Pub. No.: US 2020/0237998 A1
(43) Pub. Date: Jul. 30, 2020

(54) SYSTEM AND METHOD TO PROVIDE A CONTRAST AGENT

(71) Applicant: OAKWOOD HEALTHCARE, INC., Dearborn, MI (US)

(72) Inventor: James Richard SPEARS, Bloomfield Hills, MI (US)

(73) Assignee: OAKWOOD HEALTHCARE, INC., Dearborn, MI (US)

(21) Appl. No.: 16/756,433

(22) PCT Filed: Oct. 18, 2018
(86) PCT No.: PCT/US2018/056499

§ 371 Date: Apr. 15, 2020

Related U.S. Application Data
(60) Provisional application No. 62/574,504, filed on Oct. 19, 2017.

(51) Int. Cl. A61M 5/00; A61M 5/155; A61M 5/148; A61M 5/168; A61M 5/19; A61M 5/31; A61M 5/315

(52) U.S. Cl. CPC A61M 5/007; A61M 5/002; A61M 5/155; A61M 5/1486; A61M 5/16877; A61M 2005/006; A61M 5/3129; A61M 5/31505; A61M 2005/3132; A61M 2005/3131; A61M 5/19

(57) ABSTRACT

A liquid delivery device contains a liquid saturated with a gas in a pressure chamber pressurized to a selected amount. The liquid delivery device may be removed from the pressure chamber to deliver the saturated liquid to a volume.

### FIELD

The subject of this disclosure relates to providing a contrast agent, and particularly to providing and forming a non-dye echogenic contrast agent.

### BACKGROUND

This section provides background information related to the present disclosure which is not necessarily prior art.

A contrast agent may be provided for various imaging techniques. For example, to image a portion of a subject, such as an anatomy of a subject, absorption and/or scattering of a selected energy may occur. A contrast agent introduced into the subject may assist in enhancing absorption and/or scattering of the emitted energy. The enhancement of the absorption and/or scattering may then enhance or increase the image-ability of the selected subject.

In various techniques, an image is based upon a reflection of and/or scattering of an ultrasound wave. The ultrasound wave may impact a selected item and be reflected to be sensed by a transducer. The transducer may assist in interpreting the reflected ultrasonic wave and a processor may interpret the reflected wave, or a plurality of reflective waves to generate an image. Providing additional contrast agents within the subject may assist in insuring a high clarity image generation.

### SUMMARY

This section provides a general summary of the disclosure, and is not a comprehensive disclosure of its full scope or all of its features.

In various embodiments, a liquid delivery device may be pressurized or cause to dissolve therein a selected gas. In various embodiments, the selected gas may include substantially pure and/or sterile oxygen. The gas may be dissolved in the liquid over a selected period of time by pressurizing a container exterior to the fluid carrying container.

In various embodiments, the fluid may include sterile saline into which the gas is dissolved. The fluid may be contained within a syringe. Prior to having the gas dissolved in the liquid, the syringe may be prefilled to a selected volume, such as about 10 milliliters (ml).

The syringe may be prepared for an application, such as delivery to a subject. The syringe, therefore, may further include a covering, such as a sterile barrier. The sterile barrier and syringe may be positioned within a container. The container may then be pressurized to a selected pressure to cause a selected amount of gas to become dissolved into the fluid. At a selected time, the fluid may be a carrier fluid for the gas. Accordingly, the gas that is saturated within the fluid may be released, for example, as bubbles during a selected or appropriate delivery of the fluid.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an illustration of a syringe and a plurality of connections to a syringe;

FIG. 2 is a perspective view of a syringe in a sealed canister, according to various embodiments;

FIG. 3 is a perspective view of a syringe in a sealed canister, according to various embodiments;

FIG. 4A is a canister having a fillable volume therein, according to various embodiments;

FIG. 4B is a plain illustration of a canister having a fillable volume therein and a separate port;

FIG. 5 is a flowchart for a method of dissolving gas in a liquid;

FIG. 6 is an environmental view of a syringe connected to an intravenous system;

FIG. 7 is a plan view of a syringe that is substantially gas impermeable to hold a volume of a liquid for generating a contrast medium;

FIG. 8 is a perspective view of a pressure container with a filled container therein;

FIG. 9 is a flowchart of a method of pressurizing a container of a non-aqueous material;

FIG. 10A is a perspective view of a syringe in an open canister, according to various embodiments;

FIG. 10B is a perspective view of a syringe in a sealed canister, according to various embodiments;

FIG. 11 is a perspective view of a syringe in a sealed canister, according to various embodiments; and

FIG. 12 is a plan view of a large sleeve and formation process.

Corresponding reference numerals indicate corresponding parts throughout the several views of the drawings.

### DETAILED DESCRIPTION

Example embodiments will now be described more fully with reference to the accompanying drawings.

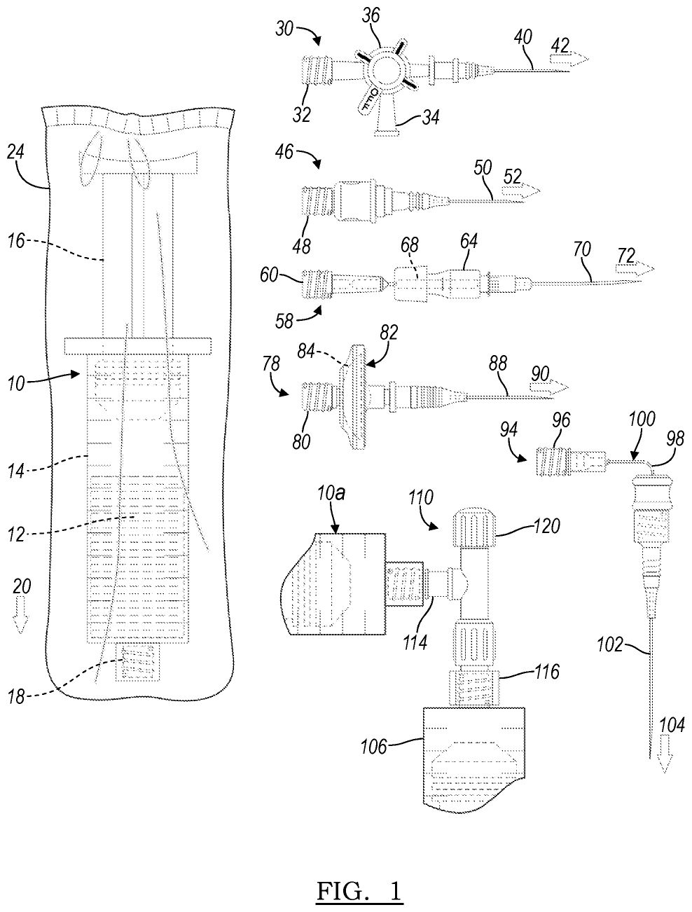

With reference to FIG. 1, a container assembly 10 is illustrated. The container assembly 10 may include a selected container and may also be referred to as a syringe. The syringe 10, or any appropriate container, may include various features for delivery of a fluid 12 to a subject. The fluid 12 may be any appropriate or selected fluid. In various embodiments, the fluid 12 may be a liquid and may include sterile saline for various applications, such as medical applications. The fluid 12 may be generally a high viscosity fluid, low viscosity fluid, and/or may be an aqueous fluid. The fluid 12 may be selectively expressed from the container 10, as discussed herein.

The syringe 10 may include various features or portions such as a barrel 14 that defines a volume in which the fluid 12 is placed and a plunger 16. The plunger 16 may be movable relative to the barrel 14 to allow for expression or delivery of the fluid 12 in a direction, such as through a tip or lock portion 18 generally in the direction of arrow 20. In various embodiments, the fluid 12 may be expressed from the container 10 by movement of the plunger 16 within the barrel 14 to drive or move the fluid 12 through the tip portion 18. Thus, the fluid 12 may, in various embodiments, be a low viscosity fluid that is expressed with pressure applied to the plunger 16.

The syringe 10 may be provided in a selected covering or a sleeve 24. The covering or sleeve 24 may be an appropriate covering, such as a sterile barrier covering. The covering 24, therefore, may allow for the maintaining of sterility of the syringe 10 and its contents even if the syringe 10 is moved from a first to a second location. The cover 24 may be positioned over the syringe 10 at a selected time to maintain the sterility of the syringe 10. In various embodiments, for example, the syringe 10 may be filled with the liquid 12 and the sleeve 24 placed over the syringe 10 and sealed.

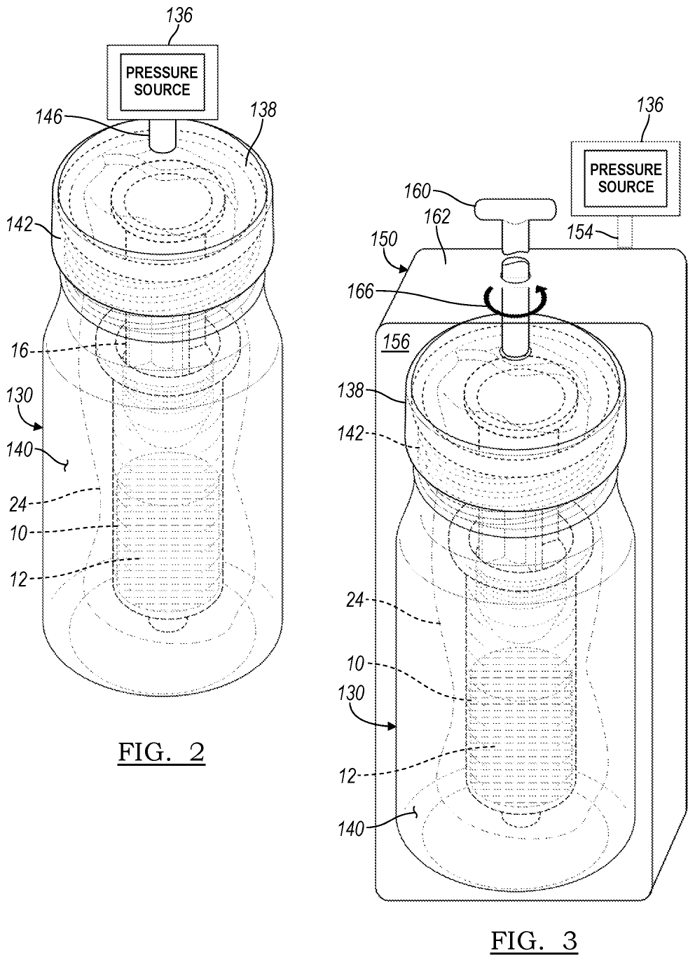

The pouch or sleeve 24 may include various characteristics, such as having a pore size to allow for sterilization of the interior of the pouch, such as the syringe 10 placed inside of the sleeve or pouch 24, due to steam sterilization. The pouch or sleeve 24, however, may also be formed of a non-porous material. For example, the sleeve 24 may be formed of a non-porous material and sealed to an exterior of the sleeve 24 with the syringe 10 therein, as illustrated in FIG. 1. Such pouches, and the contents therein, may be sterilized by selected sterilization techniques that do not use steam sterilization, such as gamma ray or gamma energy sterilization. Selected syringes and appropriate non-porous pouches may include the Praxiject saline filled syringe that is positioned in a pouch and sold by MedXL having a place of business in Quebec, Canada. The syringe within the sleeve 24, whether porous or non-porous, may be placed in the pressure container, illustrated in FIG. 2. As discussed further herein, selected pressures, such as about 45 pounds per square inch gauge (psig) to about 60 psig of oxygen, may diffuse through a non-porous portion of the sleeve 24 and into the syringe, such as in a process discussed herein. In a non-porous sleeve, diffusion of the gas within the fluid 12 within the syringe 10 may occur when the pressure exterior to the non-porous sleeve is an appropriate pressure. The diffusion to achieve a selected partial pressure within the liquid may take an amount of time, such as about 7 days to about 14 days, to achieve a selected partial pressure of oxygen within the fluid 12 within the syringe 10, such as those discussed herein.

In various embodiments, the entire assembly of the syringe 10 and the sleeve 24 may then be positioned in a selected sterilization chamber, such as in a radiation chamber. Therefore, the syringe 10 and the liquid 12 can be sterilized substantially simultaneously and be maintained until the sleeve 24 is opened. The assembly of the syringe 10 and the sleeve 10 may then be delivered for selected purposes, as discussed herein.

In various embodiments, however, the sleeve 24 and the syringe 10, at least including the volume defined by the barrel 14, may not be impermeable or substantially impermeable to selected gases. For example molecular oxygen (O₂) may be allowed to permeate through the sleeve 24 and enter the barrel 14 and/or the liquid 12. As discussed further herein, the fluid 12 may become saturated or have dissolved therein a selected volume of gas, such as molecular oxygen, for delivery at a selected time. Accordingly, the liquid 12 having a selected partial pressure of the selected gas becomes a delivery liquid after exposure to a selected pressure of the gas for a selected period of time. It is understood that variations in gas, pressure of the gas, volume of the liquid, and other variables may affect the time needed to reach the selected amount of the gas in the liquid. It is understood that any volume, such as a gas bubble or region, between the liquid 12 and the plunger 16 may be selected for ensuring a selected pressure within the syringe 10, saturation of the liquid 12, etc. For example, the free space or gas space may be 1 milliliter (ml) to 20 ml, including about 1 ml to about 10 ml, and further including about 1 ml to about 5 ml, further including less than about 5 ml.

For use of the liquid 12, once a selected saturation of gas (e.g. molecular oxygen) has been achieved, the syringe 10 may be removed from a container that has caused the saturation and/or pressurization, as discussed herein. The container may be opened quickly, just like a beverage container. After taking the syringe 10 out of the container, a user or assistant may wait for some time, such as about, a few minutes to as along as at least 30 minutes, including at least about 5 minutes to use the liquid 12 as an echo contrast injection. A gas pocket may form in the syringe 10 adjacent to the liquid 12 and may be at the same pressure (for example, about 45 pounds per square inch gauge (psi)) as the dissolved gas pressure in the liquid 12. The gas may diffuse slowly out of the syringe 10, therefore, it effectively maintains the hydrostatic pressure because of the seal on the plunger 16 is pushed back to a stop 17, such as a ledge or wall or removable locking member, at the end of the 14. Generally, a pressure on the order of greater than about 75 psi is necessary to push the plunger 16 out of the barrel 14. A cap or cover 19 on the output end 18 of the syringe 10 is generally gas-tight, so pressure is maintained for many minutes.

With continued reference to FIG. 1, the end 18 of the syringe 10 may be connected to one or more delivery assemblies. The tip 18 may include a selected connection such as a threaded connection, a Luer-Lok® connection, or other appropriate connection. A connector or tip may include a first connector tip 30 that includes a first connection portion 32 and a second connection portion 34. The first connection portion 32 may connect to the end or tip 18 of the syringe 10. The connector 30 may include a valve or stopcock assembly 36, such as generally understood in the art. Accordingly, flow from the syringe 10 may be directed and/or controlled by the stopcock 16. The fluid may be delivered through a delivery portion, such as a needle or pipette or tube 40 generally in the direction of arrow 42.

A second syringe, such as a second one of the syringes 10 may be connected to the second connection 34 to provide a second volume to be passed through the delivery tube 40. The stopcock or valve assembly 36 may be used to select which of the syringes is providing the selected fluid through the delivery needle 40. Alternatively, the valve assembly 36 may be used to direct a flow from the first inlet 32 out the connection 34 or in the connection 34 and out the connection 32. Accordingly, the valve assembly 36 is configured to allow for a selection of flow or connection of the various portions of the connection assembly 30. It is understood, as discussed herein, that other sources of fluid may be connected to the second connection 34 as well or alternatively.

In various embodiments, the stopcock 36 or other portions may form a turbulence region in the connection assembly 30. For example, the stopcock 36 may be partially closed (e.g. greater than about 50% closed). As discussed herein, the turbulence region may assist in generation of bubbles in the delivered liquid. The turbulence region may cause cavitation and/or interfere with a laminar flow of the liquid from the syringe 10. Thus, bubbles may form due to and/or at the turbulence region and exit the connector 30, or connectors according to various embodiments, as discussed herein. In various examples, bubbles were formed by manual injections (e.g. using a hand held syringe) of 10 mL saline (e.g. in over 300 experiments using a syringe sold by Becton-Dickenson Posiflush prefilled with sterile saline) into a water-filled two-liter plastic container; video of the injection was made with contrast echo videos utilizing an ultrasound system and probe (e.g. a clinical Hewlett Packard Sonos 1000 ultrasound imaging system and a 3.5 MHz ultrasonic probe). For each method for inducing turbulence with various connectors, including those discussed above, runs were performed at multiple dissolved oxygen levels and compared to injections of agitated air/saline in terms of echogenicity. In various tests, oxygen was dissolved in the sterile saline at various pressures, including about 15 psi, about 30 psi, about 45 psi, about 60 psi, and about 75 psi. Prior investigations have shown that the above described experimental procedure provides a good approximation of oxygen microbubble echogenicity in in vivo studies in anesthetized juvenile domestic swine See, Spears J R et al, A novel surfactant-free small oxygen microbubble echo contrast agent, J Amer Coll Cardiol 63:12S, 2014.

A further and/or alternative includes a connection assembly 46 may include a connection portion or end 48 to connect to the tip or end 18 of the syringe 10. A delivery needle or tube 50 may allow or to direct a fluid generally in the direction of arrow 52. The connection assembly 46 may include various features such as a turbulence causing portion, including a filter, webbing, or other material in an initial flow region 54. As discussed herein, the turbulence region may assist in generation of bubbles in the delivered liquid.

An additional and/or alternative connector 58 may also include a connection portion or region 60 to connect to the tip or end 18 of the syringe 10. The connector 58 may include various portions, such as a filter region or turbulence forming region 64. The turbulence forming region 64 may be formed of various materials, such as an open-cell foam, fibrous mesh, or other appropriate materials 68 positioned within the turbulence region at 64. As discussed herein, the turbulence region may assist in generation of bubbles in the delivered liquid. An exit tube or needle 70 may allow a fluid to exit the needle 70 generally in the direction of arrow 72.

A further connector and/or an alternative connector 78 may also be provided. The connector 78 may include a connection region 80 to connect with the port or tip 18. A connector 78 may further include a turbulence inducing region or portion 82 that may include a turbulence inducing material, such as a filter material 84 similar to those described above in the filter material 68. Selected filter materials may include ones comprised of mixed cellulose esters, such filters may include a 25 mm diameter MILLEX-GS 0.22 filter sold by Merck Millipore Ltd. As discussed herein, the turbulence region may assist in generation of bubbles in the delivered liquid. Extending from the turbulence inducing region 82 can be a tube or needle 88. Again, the fluid may exit the connector 78 in generally the direction of the arrow 90.

A further and/or alternative connector 94 may include a connection region 96 that connects to the connection tip or portion 18. A turbulence inducing region 98 may be formed as a bend or change in direction of a flow path or tube 100. As discussed herein, the turbulence region may assist in generation of bubbles in the delivered liquid. An exit or delivery tube or needle 102 may also direct the flow of the fluid generally in the direction of arrow 104.

A further and/or alternative connector may be a multi-unit connector 110 that includes a first inlet connection 114 and a second inlet connection 116. Each of the selected connections may allow for the connection of a syringe, such as a first syringe 10a and a second syringe 10b. Each of the two syringes 10a, 10b may connect to the alternative connector 110 through a “T”, “Y”, or other type of dual connection. A single outlet region 120 may be further connected to a connector, such as one of the connectors discussed above including the connector 32, or other appropriate connector.

The connector 110 may be referred to as dual or dual volume connector 110. The dual volume connector may allow two of substantially identical containers, such as the syringes 10a and 10b to allow for a substantial increase, such as a doubling of a deliverable volume, through the connector, such as the outlet 120. Accordingly plurality of syringes 10 may be connected to the single connector 110 to allow for delivery of a larger volume or continuous volume for a selected purpose. In various embodiments, for example, two of the syringes may be connected to provide double the volume to be delivered from the connector 110. The connector 110 may also allow for mixing of two different materials, such as two different liquids.

As discussed herein, the liquid 12 may be saturated to a selected amount, such as including a selected partial pressure of a selected gas. The gas may form bubbles in the liquid 12 as the liquid 12 is delivered through one or more of the selected connectors, including those discussed above. Further, each of the connectors may include one or more turbulence regions. The turbulence regions may assist in the generation or formation of bubbles for selected purposes, such as those discussed herein.

Further, a kit of one or more of the syringes with one or more of the connectors may be provided or used by a user. The kit may allow for selection of appropriate or selected connectors and volumes of the liquid 12. Thus, only one of the syringe and/or connector is not necessary.

Turning reference to FIG. 2, the syringe 10 positioned within the sleeve 24 may be further placed in a sealed container or vessel 130, which may also be referred to as a pressure vessel. Pressure vessels may be any appropriate pressure vessel, such as a Series 4600 pressure vessel sold by Parr Instrument Company having a place of business at Moline, Ill. The syringe 10 may have the prefilled liquid 12 placed within the barrel 14 of the syringe 10. The liquid 12 within the syringe 10 may be able to become saturated, at least to a certain and selected degree, due to a passage of a gas through the sleeve 24 and into the liquid 12. The sleeve 24 may be gas permeable to a selected gas, such as molecular oxygen, as may be the barrel 14 of the syringe 10. Further, it is understood, that the seal of the plunger 16 within the barrel 14 may also be gas permeable to a selected degree. For example, at an external pressure of about 60 psi a partial pressure of molecular oxygen may dissolve in the liquid to a partial pressure of about 30 psi to about 100 psi, including about 60 psi in about 72 hours. In various embodiments, the pressure vessel 130, however, is substantially gas impermeable. Selected gas impermeable containers or pressure chambers may include selected polymer and/or metal or metal alloy containers. For example, the containers that contain for consumer sale of UPTIME energy drink sold by Uptime Energy, Inc. The pressure vessel or chamber 130 may be pressurized with a selected gas from a pressure or gas source 136. The gas source 136 may be a pressurized canister, a pump, or the like. Regardless, the pressure source 136 may provide a selected gas such as sterile oxygen to the pressure chamber 130. The pressure chamber 130 may be pressurized to a selected pressure such as 30 pounds per square inch gauge (psi) to about 2000 psi, including about 30 psi to about 100 psi, and further including about 40 psi to about 100 psi, and further including about 55 psi to about 65 psi. As discussed above, the gas used to fill the pressure chamber 130 may then permeate the syringe 10 and dissolve in the liquid. At a selected solution, such as about greater than 2 atmospheres (about 30 psi) partial pressure of the selected gas (such as medical grade oxygen gas) in the saline, the liquid 12 in the syringe 10 may be selected to be a delivery liquid, as discussed herein.

The pressure vessel 130, once pressurized from the pressure source 136 to the selected pressure, as noted above, remains sealed with an appropriate sealing member, such as a sealing cap 138. The sealing cap may engage the container 130 to enclose or seal a volume 140 within the container 130. The cap 138 may be fit to the container 130 in a selected manner, such as being threaded onto a neck 142 through an engagement of threads between the cap 138 and the neck 142. The cap 138 may in addition or in the alternative, however, may be press-fit onto the neck 142 such as with a moldable plastic, metal, or the like. In addition to the use of a twist off cap, a full aperture ring pop-off aluminum beverage end, for example including caps sold by Crown Holdings, Inc. could be used to seal the oxygen pressurized vessel.

Regardless of the mechanism, however, the cap 138 generally substantially seals the container 130 and the volume 140 within the container 130. The volume 140, therefore, remains pressurized at the selected amount, or within a selected variance of the selected amount, for a selected period of time. In various embodiments, the pressure within the volume 140 may be maintained at the selected pressure within about 5% to about 10% for a selected period of time, such as several minutes, to several hours, days, or longer. In various embodiments, the pressure vessel 130 may maintain the selected pressure for at least about 2 months to about 6 months. In experiments, a container including the container in which UPTIME energy drink is delivered as sold by Uptime Energy, Inc. included a less than about 0.05 gram change in mass over a period of two months. Thus, the change in gas volume and/or pressure is less than about 5% over the same period. Various sealing members may be fitted to the pressure vessel 130 such as a metal press on lid and/or a polymer twist on lid. It is understood, however, that sealing the pressure vessel 130, such as with the cap 138, may cause the pressure to be maintained at a selected amount within the pressure vessel 130 at a selected period of time, which may be substantially indefinite.

The cap 138 may also allow the pressure source 136 to be removably connected to the volume 140. For example, a delivery port or connection 146 is formed to extend from the cap 138. The pressure source 136 may removably connect with the cap 138 via the connection 146 to allow pressure to be delivered to the container 130. The container 130, therefore, can be maintained at the selected pressure to ensure a solution of a selected amount of the gas within the liquid 12 within the syringe 10 for a selected application, as discussed further herein.

Turing reference to FIG. 3, the pressure container 130 may be pressurized in an alternative and/or additional manner. For example, the pressure container 130 may be placed within an external or secondary pressurization chamber or unit 150. The secondary pressurization unit 150 may be connected with the pressure source 136 through a selected connection, such as a connector 154. The pressure source 136, therefore, may pressurize an internal volume 156 of the secondary pressure container 150. The pressure chamber 130 may be initially unsealed, while containing or holding the sleeve 24 with the syringe 10 therein, including the contained liquid 12.

The pressure chamber 130 being unsealed when the secondary pressure chamber 150 is pressurized from the pressure source 136 thereby also pressurizes the pressure chamber 130 to the selected pressure. The pressure source 136 may pressurize the external volume 156 of the secondary chamber 150 to a selected amount with a selected material, such as a selected gas including molecular oxygen or other appropriate gas. As the pressure chamber 130 is not sealed, the pressure chamber 130 is also exposed to the pressure within the secondary pressure volume chamber 150 as is the syringe 10 within the pressure chamber 130.

After the secondary pressure chamber 150 is pressurized to the selected amount, such as the amount discussed above, the cap or sealing member 138 may be sealingly connected to the pressure chamber 130. For example, a closing mechanism 160 may extend through an external wall 162 of the secondary pressure chamber and engage the cap 138. As discussed above, the cap 138 may threadibly engage a neck 142 of the pressure chamber 130. Accordingly, the connection member 160 may rotate the cap 138 in a selected direction, such as the direction of the arrow 166, to threadibly engage the cap 138 on the neck 142. Once the cap 138 is sealingly engaged onto the pressure chamber 130, the pressure of the secondary pressure chamber 150 is maintained within the pressure chamber 130, such as the internal volume 140 of the pressure chamber 130 over a period of time similar or identical to that as discussed above. Once sealed, the pressure chamber 130 may then be removed from the secondary pressure chamber 150 and stored in a selected manner for later use, as discussed herein.

Accordingly, the pressure chamber 130 may be filled directly or pressurized directly at a selected pressure with a selected material, such as molecular oxygen, via the connection 146 through the cap 138 or other appropriate connection. Alternatively, or in addition thereto, the pressure chamber 130 may be pressurized and filled with a gas by being placed in the secondary chamber 150, and then the secondary chamber 150 being pressurized from the pressure source 136 to a selected degree or with the selected amount of material and then sealed within the pressure chamber 150. Thus, the pressure chamber 130 may be pressurized and/or filled with the selected material recording an appropriate embodiment to allow for solution of the selected material, such as molecular gas, within the liquid 12 contained within the syringe 10.

The pressure chamber 130 and/or the secondary vessel 150 may be pressurized to a selected pressure, as discussed herein. The pressure chamber 130 and/or the secondary vessel 150 may be formed of a selected material and/or coated with a selected material to maintain the selected pressure or within a variance thereof, such as about 5% to about 10%, for a selected period of time, such as long enough to saturate and maintain saturation of the liquid for a selected period of time. For example, containers formed of aluminum, PET, etc. may be used as the pressure chamber 130 and/or the secondary vessel 150. Further, the pressure chamber 130 may be sized to hold one, two, or more of the syringes 10 and the volume of gas added to pressurize the pressure chamber 130 may be selected to be enough to saturate the liquid 12 in all of the syringes 10 within the pressure chamber 130.

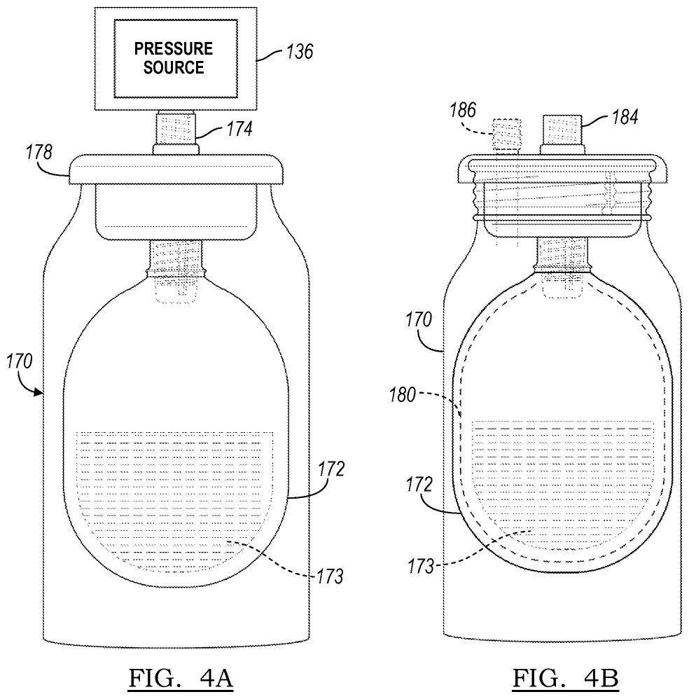

According to various embodiments, a volume of a liquid may be directly pressurized or filled to a selected partial pressure with a selected gas, such as molecular oxygen. For example with reference to FIG. 4A, an external chamber or container 170 may have contained therein a flexible member or container 172. The flexible member 172 may be filled with the selected material, such as saline or sterile saline according to appropriate formulations. A pressure source, such as the pressure source 136 may be connected with a connector 174 through a portion of the container 170, such as a lid 178. The connector 174 may be connected directly to the inner chamber 172. Thus, the pressure source 136 may directly pressurize the container 172 within the external container 170, such as to a pressure as discussed above. This allows a material, such as saline, positioned with the inner chamber 172, to be pressurized to the selected degree as discussed above. In such an instance, the liquid within the inner chamber 172 may be caused to have dissolved therein the material that is used to pressurize the inner chamber 172, such as the molecular oxygen. According to various embodiments, therefore, once the selected amount of solution of the gas within the liquid in the inner chamber 172 is reached the container 170 can be stored for a selected use, such as withdrawal of the saturated liquid from the inner chamber 172.

According to various embodiments, as illustrated in FIG. 4B, the chamber 170 including the inner chamber 172 may further include a secondary inner chamber 180 within the first inner chamber 172. The secondary inner chamber 180 may directly contain the liquid 173, such as the saline including sterile saline as discussed above. The inner chamber 172 may, therefore, be placed between the inner chamber 180 and the chamber 170.

The secondary inner chamber 180 may be connected to a port 184 that allows for access of the material within the secondary inner chamber 180, such as the liquid 173. A pressurization port 186 may allow for pressurization of the exterior container 170 by connection to a selected gas or pressure source, such as the pressure source 136. The external container 170 may, therefore, be pressurized, similar to the external container or secondary pressure chamber 150 as discussed above. The inner chamber 172 and secondary inner chamber 180 may not be entirely gas impermeable, therefore as discussed above, the pressurization of the outer chamber 170 may allow for a transfer of gas to the liquid 173 over a selected period of time rather than directly filling the inner chamber 180 with a saturated liquid. By pressurizing the external chamber 170, rather than the inner chamber 172 or the secondary inner chamber 180 directly, may still allow for solution of the gas within the liquid 173 at a selected rate over a selected period of time.

According to various embodiments, therefore, a liquid may be formed to include a selected amount of dissolved gas for a selected application. As discussed above a syringe, such as the syringe 10, may be prefilled with a selected liquid. The syringe 10 may be placed in a container that is then pressurized to a selected pressure and gas may be allowed to transfer into the liquid within the syringe. The syringe may be positioned or covered with a sleeve, which may be also gas permeable, to a selected degree, thus allowing a selected amount of gas to be dissolved within the liquid. The sleeve, however, as noted above may allow for maintenance of a sterility of the syringe 10 until immediately prior to use.

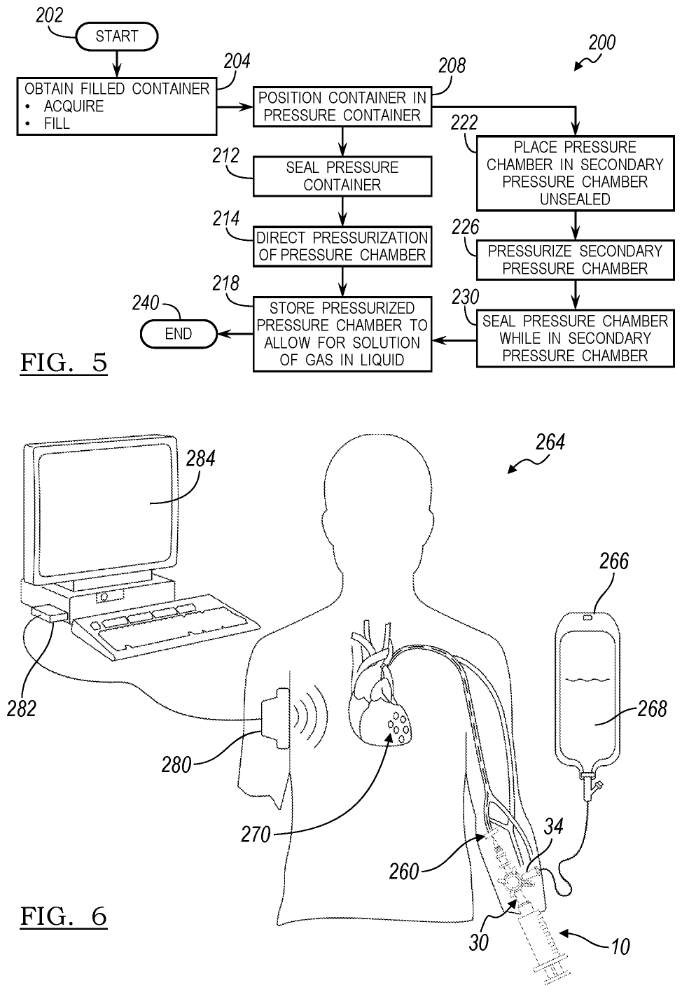

With reference to FIG. 5 a selected container including a liquid or appropriate material may be caused to have dissolved therein a gas. As noted above, for example, the syringe 10 may pre-loaded with a selected volume of a liquid, such as saline, including sterile saline, and then may be placed in the pressure chamber 130. A method 200 to dissolve a selected amount of gas in the liquid 12 is illustrated. The method 200 may include starting in start block 202. Moving to block 204, obtaining a filled container may be performed. Obtaining a filled container may include purchasing or acquiring a prefilled syringe 10. The syringe 10 may be filled with the liquid, such as sterile saline including a selected solute such as sodium chloride. In various embodiments, the syringe 10 may be pre-filled from a vendor, such as Becton Dickenson having a place of business in Franklin Lakes, N.J. The vendor may provide the saline within the syringe 10 in a substantially sterile manner and may include the sleeve 24. An alternative, or in addition thereto, the syringe 10 or other appropriate container may be filled by a user. The user may fill the syringe 10 in any appropriate manner, such as drawing a selected liquid into the syringe 10. Accordingly, the obtained container may be filled in any appropriate manner or be obtained pre-filled from a selected vendor or source. In addition, it is understood that the container may be filled by a second user at the direction of the first user without purchasing a pre-filled container.

Once the filled container is obtained, the filled container is positioned in a pressure container or chamber in block 208. As discussed above, the pressure chamber may include the pressure chamber 130 into which the syringe 10, with or without the sleeve 24, is placed. Once the filled container is positioned in the pressure chamber the pressure chamber may be pressurized. As discussed above pressurizing the pressure chamber, such as the pressure chamber 130, may include direct pressurization of the pressure chamber 130 by first sealing the pressure chamber in block 212 and then at direct pressurization of the pressure chamber in block 214. As discussed above, the sleeve 24 and/or the syringe 10 may be gas permeable at a selected rate. In various embodiments, the permeability may allow 10 ml of sterile saline to be saturated to at least a partial pressure of about 30 psi of molecular oxygen within about 24 hours to about 72 hours. Accordingly, once the pressure chamber 130 is pressurized the gas may pass into the liquid 12 over a selected period of time.

Once the pressure chamber is pressurized in block 214, storing the pressurized chamber in block 218 to allow dissolving of the gas in the liquid for a selected or appropriate amount of time may occur in block 218. Storing the pressure chamber with the syringe 10 therein may be at any selected amount of time, such as for a short period of time including minutes or hours, or for a longer period of time including days or weeks. Nevertheless, storing the pressurized pressure chamber may include storing the pressurized pressure chamber for an amount of time in and such a matter (e.g. maintaining a seal of the pressure chamber) to allow for a selected solution of the gas within the liquid of the syringe 10. As noted above, the liquid 12 within the syringe 10 may become or be selected to be a delivery liquid, such as for generation of bubbles as discussed herein, at a time when there is dissolved therein oxygen at about 30 psi partial pressure. It is understood, however, that the delivery liquid may include about 30 psi to about 70 psi, including at least about 30 psi to about 40 psi partial pressure of oxygen.

As further noted above, for example with reference to FIG. 3, the pressure chamber 130 may be placed in a secondary pressure chamber in an unsealed state in block 222. The unsealed pressure chamber and the secondary pressure chamber may allow for a gas to enter the pressure chamber 130. Accordingly, the secondary pressure chamber may then be pressurized to a selected amount, such as that discussed above, in block 226. Once the secondary pressure chamber is pressurized the pressure chamber 130 may be sealed while in the secondary pressure chamber in block 230. Again once sealed, the sealed pressure chamber, such as the pressure chamber 130, may then be stored for a selected period of time in block 218. It is understood that the pressure chamber 130 may be removed from the secondary pressure chamber, such as the secondary pressure chamber 150, as illustrated above once the pressure vessel 130 is sealed at the selected pressure. Nevertheless, as noted above, the gas that is used to pressurize the secondary pressure chamber and/or the pressure chamber 130 may then move into a liquid within the container, such as the syringe 10, to be dissolved in the liquid.

The container may then be prepared for use at a selected period of time and the process may end in block 240. Once the selected amount of solution of gas within the liquid, such as the liquid 12, has been achieved the container 10 may be used for a selected purpose. The achievement of the selected amount of dissolved gas may be based on a passage of time. Therefore, the pressure vessel 130 may have a date of preparation affixed thereto or a date of first use affixed thereto. Also, weighing the pressure vessel 130 may assist in ensuring that the mass of gas has not escaped over a passage of time.

Use of the liquid 12 from the syringe 10 may be at a selected time. As noted above, the syringe 10 may be stored in the pressure container for a selected period of time after the END 240. Further, once selected to use the liquid 12 from the syringe 10, the syringe 10 may be removed from the pressure vessel 130 for a selected period of time. As noted above, the gas (e.g. molecular oxygen) diffuses slowly from the liquid 12. Further, the syringe 10, and/or containers according to various embodiments, may maintain a selected pressure within the syringe 10. Thus, the syringe 10 may be removed from the pressure vessel 130 and held at ambient pressure for a period of time, also referred to a preparation time. The preparation time may be selected to be various periods of time, such as about 1 minute to about 30 minutes, including about 5 minutes. The preparation time may be based on materials and construction of the syringe 10, amount of contrast selected, or other variables.

For example, with reference to FIG. 6, the container 10 may be used as a contrast agent for a selected procedure. For example the syringe 10 may be connected to an intravenous connection 260 that is connected to a subject or patient 264. As illustrated in FIG. 6, one or more of the connectors, including the connector 30, may be used to connect the syringe 10 to the intravenous connection 260. The intravenous connection 260 may also allow for introduction of other liquids into the subject 264, such as from an IV bag 266 that includes a selected material, such as sterile saline 268 that may be interconnected with the connector 30, such as through the second connection 34.

The intravenous connection 260 allows for the selected liquid 12 from the syringe 10 to be delivered to a portion of the patient 264, such as a heart chamber 270. Upon delivery of the liquid 12 from the syringe 10 through the connector 30 a selected bubble or plurality of bubbles may be formed that are delivered to the heart chamber 270. Formation of the bubbles may be due to a nucleation event within the syringe 10 and/or as the liquid exits the syringe 10. In various embodiments, the interference or turbulence regions, as discussed above, formed within the connectors, e.g. connector 30, cause bubble nucleation and formation. The gas dissolved within the liquid 12 at the selected pressure, as discussed above, fills the nucleated bubbles that nucleate at the turbulence or in the turbulence that are then delivered to the heart chamber 270. The formed bubbles are generally of a selected average size of about 10 micrometers to about 100 nanometers and are substantially or completely filled with the gas (e.g. sterile molecular oxygen) that has saturated the liquid 12 within the pressure chamber 130.

The bubbles act as a contrast agent which may allow for better imaging of the heart chamber 270 with an ultrasound system including, an ultrasound transducer 280 and associated ultrasound processing system 282. The ultrasound processing system 282 may generate an image that is displayed on a display device 284 according to appropriate mechanisms. Ultrasound systems may include SONOS™ Ultrasound Imaging System sold by Hewlett-Packard.

Accordingly the liquid 12 from the syringe 10 may be provided to the subject 264 to generate a contrast within the subject 264 at an appropriate or selected location. The bubbles may be generated due to the dissolved selected gas, such as molecular oxygen, within the saline. Both the saline may be sterile, due to its storage in the syringe 10, and the gas in the bubbles may also be sterile due to the provision of the selected pressure source, such as sterile molecular oxygen from the pressure source 136. Accordingly, the contrast agent (e.g. the bubbles) and the delivery thereof may be substantially sterile to the subject 264. Moreover, the bubbles having the selected size range, as noted above, may dissolve at a selected rate within the subject 264 as they are full of substantially pure oxygen. Accordingly the bubbles may be the only contrast agent (such as an echo-contrast agent) for selected imaging, such as ultrasound imaging. Further, the bubbles may also provide oxygen to the subject 264 in a selected manner without the requirement of additional contrast dyes or other chemicals.

The syringe 10, including the oxygenated or oxygen saturated fluid, may be delivered to the intravenous line or portion 260 for generating the bubbles 270 as a contrast agent, as discussed above, or for various additional purposes. For example, the intravenous connection 260 may be a venous catheter connection, such as a central venous catheter, dialysis catheter, or the like. The venous catheters may be connected to the subject 264 for various purposes, such as general or repeated blood draw or infusion, dialysis, or the like. In various instances, cleaning or flushing the catheters may be performed with the fluid in the syringe 10.

As discussed above, the fluid in the syringe 10 may be saturated, such as supersaturated, with a selected gas, such as oxygen. As discussed above, the gas, such as oxygen, may be at a selected or determined partial pressure within the liquid that is within the syringe. The oxygen, such as in combination with the liquid, may serve to clean the catheter such as the intravenous connection 260, through the generation of the bubbles at or near the catheter connection and movement of the bubbles, such as agitation, therein. Accordingly, the placing or passing of the fluid through the intravenous connection may assist in cleaning the intravenous connection or catheter.

In addition, the delivery of oxygen may assist in aerobic or in enhancing an aerobic environment within the intravenous connection. The introduction of oxygen may assist in the destruction or removal of bacteria by enhancing oxygen dependent leukocytes killing and removal of bacteria. Additionally the enhanced aerobic environment, due to the introduction of the gas saturated liquid, may have a direct bactericidal effect and/or ensure a static bacteria, if present, and limit or eliminate growth. Accordingly, the syringe 10 may be used, as illustrated in FIG. 6, to deliver the liquid for generation of bubbles for a contrast agent and/or cleaning of an intravenous line or assisting and reducing bacterial growth or removing bacteria, as discussed above.

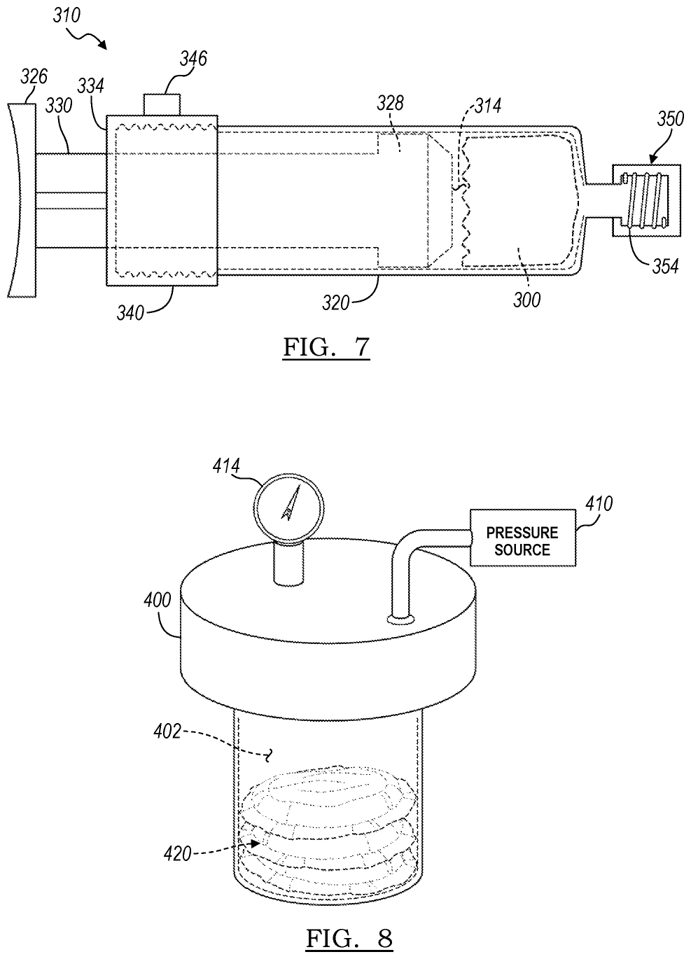

As discussed above, the syringe 10 may include the liquid 12 that is saturated to a selected amount, as discussed above, at least by dissolving a gas in the liquid 12 over a period of time due to a transfer of gas from the pressure vessel 130 into the syringe 10 and the liquid 12. It is understood, however, that a delivery container according to various embodiments, may be provided with a liquid 300, as illustrated in FIG. 7. The liquid 300 contained in the delivery container may be within a syringe 310 wherein the liquid 300 already has dissolved therein a selected amount of the gas, such as molecular oxygen. The amount of gas dissolved in the liquid 300 may be molecular oxygen dissolved at about 15 psi to about 75 psi.

The syringe 310 may be provided with selected materials that are substantially gas impermeable, such as impermeable to molecular oxygen. Further, the syringe 310 may be sealed, according to various embodiments and various features, including those discussed further herein, to ensure or substantially ensure maintaining the selected partial pressure or pressure of molecular oxygen within the liquid 300 over a selected period of time. As discussed above, the selected period of time may be in the order of hours, days, months, or longer. Further, it is understood, that the syringe 310 may be filled with the liquid 300 that does not have a gas dissolved therein. With the liquid 300 in the syringe 310 may be provided with a charge of the gas, such as in an air or gas pocket 314, at a selected pressure. For example the gas pocket 314 may be filled at about 15 psi to about 100 psi to allow oxygen to diffuse into and be dissolved in the liquid 300 over a selected period of time.

The syringe 300 may include a syringe wall or barrel 320 of a selected material that is substantially gas impermeable. The syringe wall 320 may be formed of selected materials such as Polyethylene Terephthalate (PET) and/or PET Composites. It is understood that other appropriate polymer and polymer composites, particularly those that are biocompatible, may be used to form the barrel 320. In addition, various other materials may be used to form the barrel 320 such as various multi-layer polymers (where in each layer may be the same or a different polymer) either alone or in combination with aluminized polymer layers and/or aluminum foil layers. Various systems may include features such as those included in the Power Pouch sold by the Power Container Corporation having a place of business in Somerset, N.J.

Further various materials or surfaces may be coated with selected coatings to assist in maintaining a gas impermeable wall. For example, a polymer may be coated with a silicon dioxide. Various materials include a FlexiMed coating of silicon dioxide sold by Hoffman Neopac AG having a place of business in Switzerland. Further it is understood that the barrel 320 may be formed of various other gas impermeable or substantially gas impermeable materials such as metal or metal alloys (e.g. aluminum or aluminum alloys), ceramics, and the like.

The syringe 310 may include a plunger 326 that includes a stopper or sealing feature or member 328 positioned within the barrel 320. The plunger or sealing member 328 may be formed of selected materials that are also substantially gas impermeable, such as butyl rubber. It is further understood that various additional sealing features or members, such as one or more O-rings, may be positioned around a shaft 330 of the plunger 326 to assist in sealing the barrel 320 and the liquid 300 and/or the gas 314 in the barrel 320. The barrel 320 may be sealed at or near a first end 334 by the plunger 326 or a plunger assembly including the plunger seal 328 and/or sealing members positioned around the shaft 330 to engage the barrel 320.

An additional threaded or locking member 340 may engage the plunger assembly 326 to assist in holding the plunger assembly 326 within the barrel 320. The liquid 300, including the dissolved gas therein, may be pressurized within the barrel 320. Accordingly the locking feature 340 may hold the plunger assembly 326 within the barrel 320 to maintain the pressure within the barrel 320 after filling the barrel 320 to a selected volume. Maintaining the plunger within the barrel 320 additionally maintains the dissolved gas within the liquid 300. Various quick release or release mechanisms, such as a release valve or release button 346, may allow for rapid axial movement of the plunger 326 along a longitudinal axis of the barrel 320 such as toward a second end 350. At or near the second end 350 may be a connection feature or portion 354 similar to the connection or end 18, as discussed above, of the syringe 10. Accordingly, one or more of the connectors discussed above may be connected to the end or tip 354 to allow for a selected use of the syringe 310 as discussed above.

The syringe 310 may be used to create or maintain a pressure within the barrel 320 of an appropriate amount. For example, a pressure formed within the barrel 320 may be allowed 100 psi to about 300 psi. Various syringe assemblies may include the BASIXCOMPAK™ inflation device sold by Merit-Medical Systems, Inc. having a place of business at Utah, USA. Various other screw type or high pressure syringes may include the Encore™ inflator sold by Boston Scientific Corporation and/or the Angioflator sold by Taha Medikal, and others. The various syringes, including the syringes as embodied in the syringe 310, may include a screw or threaded feature to allow for creation and/or maintaining of a high pressure within the barrel 320 over a selected period of time. The syringe 310 may also include the quick release or axial release 346 to allow for the plunger assembly 326 to move axially within the barrel 320, such as for expression or delivery of the liquid 300.

According to various embodiments, the syringe 310 may be filled at a selected period of time and maintained filled with the selected pressure in the syringe 310 without the need for an additional and/or external pressure vessel 130. The syringe 310 formed of a selected substantially gas impermeable material and including various features, such as the thread locking mechanism 340 to maintain the plunger assembly 326 within the barrel 320, may maintain the selected pressure within the barrel 320 to ensure or select an amount of the gas within the fluid 300. It is understood that the syringe 310 need not include the threaded mechanism but may include other features, such as only an O-ring or locking O-ring, or other sealing feature to maintain the plunger assembly 326 within the barrel 320.

Further, it is understood, that the syringe 310 may be filled at a selected time near, such as within minutes or hours prior to use in a selected procedure. According to various embodiments, the syringe 310 may be filled with the selected volume of the liquid 300 and pressurized, such as through the plunger assembly 326 and/or the tip end 354 to a selected pressure. The syringe, being substantially gas impermeable, it may be maintained at a selected amount of time to allow for a selected volume of gas to be dissolved within the liquid 300. The liquid 300 may then be delivered to a selected subject, such as discussed above and illustrated in FIG. 6, including for generation of bubbles for an echogenic contrast. Accordingly, the syringe 310 need not be maintained in a substantially high pressure or selected pressure environment, such as within the pressure vessel 130, to allow for saturation of the liquid 300 to a selected amount. The syringe 310, at least in part due to its gas impermeability, may allow for saturation of the liquid 300 to a selected degree based upon the features and characteristics of the syringe 310 alone. Nevertheless, the liquid 300 may be delivered for an appropriate procedure, as discussed above.

The pressurized fluid or gas may provide pressure to any appropriate pressure container, such as a pressure container 400 illustrated in FIG. 8. The pressure container 400 may be pressurized to a selected pressure within the container, such as in an interior volume 402 thereof. The pressure container 400 may be any appropriate pressure container, such as a Series 4600 pressure vessel, as discussed above. Generally, pressure source 410 may be used to provide a volume of a fluid, such as medical grade or sterile oxygen gas to the pressure vessel 400, in particular to the interior volume 402 thereof. Selected instruments or connections may also be made to the container 400, such as a pressure gauge 414. The pressure gauge 414 may be an analog, digital, or other connection gauge to the pressure container 400. The pressure gauge 414 may be used to determine a pressure of the interior volume 402 of the container 400.

In various embodiments, a selected item or package may be placed in the interior volume 402. For example, a viscous material containing container 420 may be placed in the volume 402. The container 420 may be selected materials, such as packets of white petrolatum (e.g., white petroleum jelly). In various embodiments, the packets may be of a selected size, such as a generally small size including about 5 grams of the material. The material may be a substantially viscous material or have a high viscosity, such as a generally cream or petroleum jelly consistency. Exemplary packets may include white petrolatum 5 gram foil packs (e.g., item number 1140 white petrolatum-5 g foil packs (HCPCS code A6250) sold by Dynarex having a place of business in Orangeburg, N.Y.). The foil packs as may have a selected volume or mass, such as about 5 grams, of white petrolatum therein. The foil packs may include a foil liner of aluminum or selected metal foil that may be further surrounded by a polymer or cellulose packaging. The packages are generally small in size and one or a plurality of packages may be placed within the internal volume 402 of the pressure container 400. The pressure container 400 may be pressurized from the pressure source 410 to the selected pressure for a selected period of time.

For example, the pressure source 410 may be able to provide a selected volume and/or pressure of medical grade oxygen to the pressure container 400. In various embodiments, the pressure source 410 may be able to provide the medical grade oxygen at a pressure of about 900 psig to about 1000 psig (about 61 bars to about 68 bars). The pressure of the medical grade oxygen may be maintained in the internal volume 402 of the pressure vessel 400 for a selected period of time such as about 96 hours to about 192 hours, further including about 120 hours to about 168 hours, further including about 5 days to about 7 days. After the selected period of time the packet 420 including the selected material may have a selected volume or an appropriate volume of the oxygen diffused therein. In various embodiments, the foil packs 420 may be substantially impermeable to gas (such as oxygen) at atmospheric pressure (i.e. about 1 atmosphere). However, at selected high pressures, such as about 900 psig to about 1000 psig, a selected amount of the oxygen diffuses into the material, such as the petrolatum, within the packaging 420. According to various embodiments, at the selected pressure and for the selected period of time, as discussed above, about 0.2 milliliters (ml) to about 0.5 ml of oxygen is infused per about 2 ml per gram of material and further including about 1 ml of oxygen per gram of white petrolatum within the packet 420.

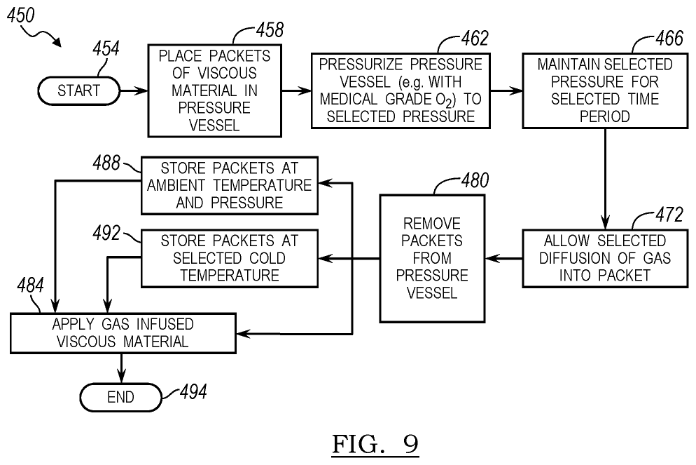

With continued reference to FIG. 8 and additional reference to FIG. 9, a process of diffusing gaseous oxygen into a substantially viscous material, such as white petrolatum, is illustrated in the flowchart 450. The process may start in start block 454. The process may then include placing packets in a pressure vessel in block 458. The process then includes pressurizing pressure vessel to a selected pressure with a selected gas in block 462. As discussed above, the selected gas may include medical grade gaseous oxygen (i.e. O₂) to a selected pressure, such as about 900 psig to about 1000 psig. It is understood that an alternative selected pressure may also be provided, such as about 700 psig to about 1200 psig and further including about 500 psig to about 2000 psig.

The pressure is then maintained in the pressure vessel for a selected period of time. The selected period of time, as discussed above, may be an appropriate period of time including about 3 days to about 10 days, and further including about 5 days to about 7 days. Pressurizing the pressure vessel to the selected pressure in block 462 and maintaining the pressure for the selected period of time at block 466 allows for a selected diffusion of the gas into the packet in block 472. As discussed above, the amount of oxygen within the packet may be about 1 milliliter per gram of the viscous material, such as about 0.1 ml to about 1 ml of oxygen per gram of petrolatum within the packet 420.

After the selected amount of gas is diffused into the packets or has been allowed to diffuse into the packets in block 472, the packets may be removed from the pressure vessel in block 480. The packets, once removed from the pressure vessel in block 480, may then be applied to a selected subject, such as a patient (e.g., a human patient) or any other appropriate subject. It is also understood that the material within the packets may be any appropriate selected viscous material such as petroleum grease, polymer oils, or the like. Moreover, the gas used to pressurize the pressure vessel may be any appropriate gas and need not to be gaseous oxygen, but may include other gases, such as noble gases (e.g., argon) or other selected atmospheric gases such as nitrogen, or the like. Accordingly, the application of the gas infused viscous material may be to any appropriate subject for selected purposes.

In various embodiments, the gas infused viscous material includes white petrolatum. In the gas infused petrolatum, the gas may form bubbles or pockets within viscous material of a selected size, such as about 4 micrometers to about 20 micrometers in diameter. The pockets may form especially upon opening of the packet 420.

After removing the packets from the pressure vessel in block 480, the packets may be stored at an ambient temperature and pressure in block 488. Over a selected period of time, such as about two months, the amount of oxygen maintained within the packets after being stored in an ambient temperature and pressure (e.g., about 20° C. to about 24° C.) and atmospheric pressure is reduced by about 60%. After storing the packets for a selected period of time at an ambient temperature and pressure the viscous material may then be applied to a selected subject in block 484.

Alternatively, or in addition thereto, the packets may be stored at a selected cold temperature in block 492. For example, the packets may be stored at a temperature of about −4° C. Upon storing the packets at about −4° C. for the same period of time, such as about two months, only about one-half or 50% of the oxygen is released or escaped from the packet 420. After storing the packets at the selected cold temperature in block 492, the selected viscous material may also be applied to a subject in block 484. The process or method may then end in block 494.

The volume or amount of the gas diffused into the packet 420 during the method 450 may be increased such as by increasing the pressure of the pressure vessel 400 and/or heating the internal volume 402 of the pressure vessel 400. Also lowering the temperature of storage in block 492 and/or maintaining at least a slightly elevated (e.g., about 2-3 times standard atmospheric pressure) of the gas used to pressurize the pressure vessel in block 462, the maintained amount or volume of the gas within the viscous material within the packets 420 may be increased.

The packets 420, including the viscous material, may be infused with a selected gas, such as infusing white petrolatum with medical grade oxygen through diffusion of the oxygen into the packets and the oxygen is maintained at a selected or effective amount of concentration for an extended period of time after removing the packets 420 from the pressure vessel. In various embodiments, the application of the oxygen infused petrolatum may provide a surface application of an oxygenated material to a selected subject, such as a human subject during a healing process.

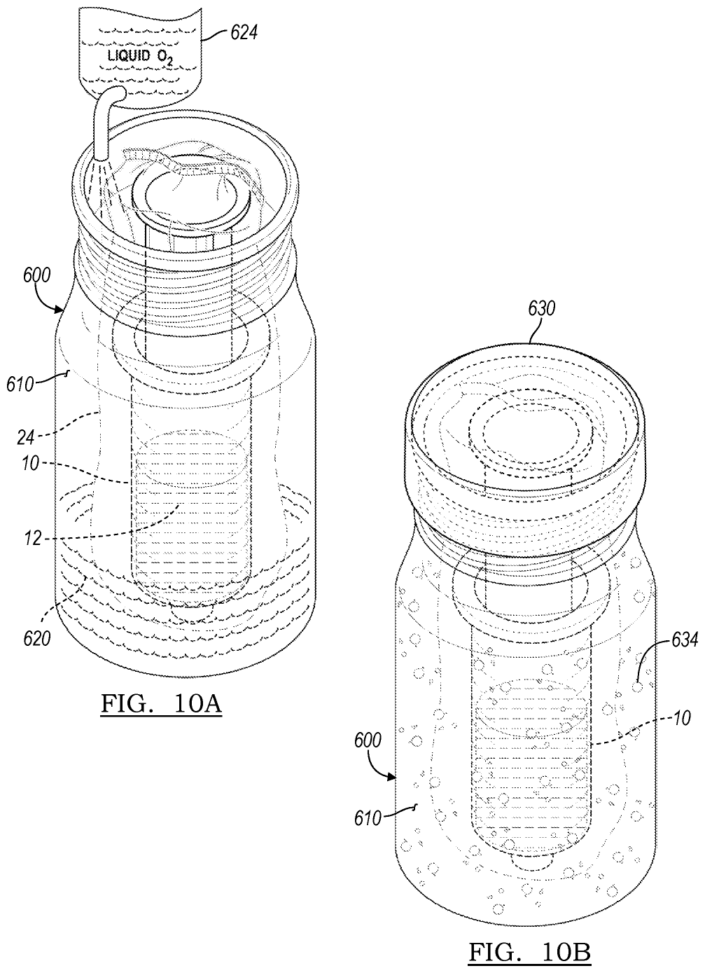

With reference to FIG. 10A and FIG. 10B, a container or vessel 600 is illustrated. The vessel 600 may have an interior volume 610 into which the sleeve or pouch 24 containing the syringe 10 may be positioned. The syringe 10 may include the fluid 12 within the syringe 10. It is understood that the syringe 10 may include additional features, such as that discussed above, which are not described again here in detail.

To pressurize or dissolve gas within the fluid 12, as discussed above, the container 600 may be pressurized from a selected pressure source. Accordingly, the interior volume 610 may be pressurized with a selected gas, such as oxygen or pure oxygen. Under various conditions, a material that is a gas at standard temperature and pressure (STP, i.e. 1 atmosphere of pressure and 0 degrees Celsius) may be a liquid or a solid. In various embodiments, for example as illustrated in FIG. 10A, a selected mass or volume of liquid or solid (i.e. first) form of a material (e.g. oxygen or nitrogen) 620 may be placed within the container 600 from a selected source, such as a liquid gas source 624. The liquid gas source 624 may deliver the first form material 620 into the container 600 either through a lid or through an open end of the container 600. Certain liquid delivery systems include the introduction of liquid nitrogen to barrier systems such as the NITRODOSE® liquid nitrogen delivery machines provided by Vacuum Barrier Systems having a place of business in Woburn, Mass.

After the first form material 620 is delivered to the container 600, a lid 630 may seal the container 600, such as gas tight. Under standard atmospheric pressure and temperature, the first form material 620 may phase change (e.g. vaporize or sublimate) into a second form of the material, such as a gas 634. The gas 634 is maintained within the interior 610 of the container 600 due to the container 600 and the lid 630 positioned on the container.

The gas pressure of the gas 634 within the container 600 may be based upon the volume or mass of the first form material 620 and the volume of the interior 610 of the container 600. The first form material 620, therefore, may be selected based upon the volume of the interior 610 and the selected or desired pressure to be developed within the interior 610 of the container 600. In various embodiments the syringe 10 may include the fluid 12 with a selected internal pressure of oxygen, such as about 45 prig at standard pressure and temperature. A two gram mass of liquid of oxygen as the first form material 620 may be delivered to a selected volume, such as the container 600 having the internal volume 610 of about 355 ml would achieve about 3 atmospheres of pressure of O₂ as the first form material 620 vaporizes to the gas 634.

Once the first form material 620 vaporizes to the gas 634 within the container 600, the container 600 may maintain the selected pressure within the liquid 12 and/or increase the pressure of the gas within the liquid 12, similar to what is discussed above when applying high pressure to the container 600. The first form material 620 may, therefore, be the same material that is to pressurize or form a partial pressure within the liquid 12.

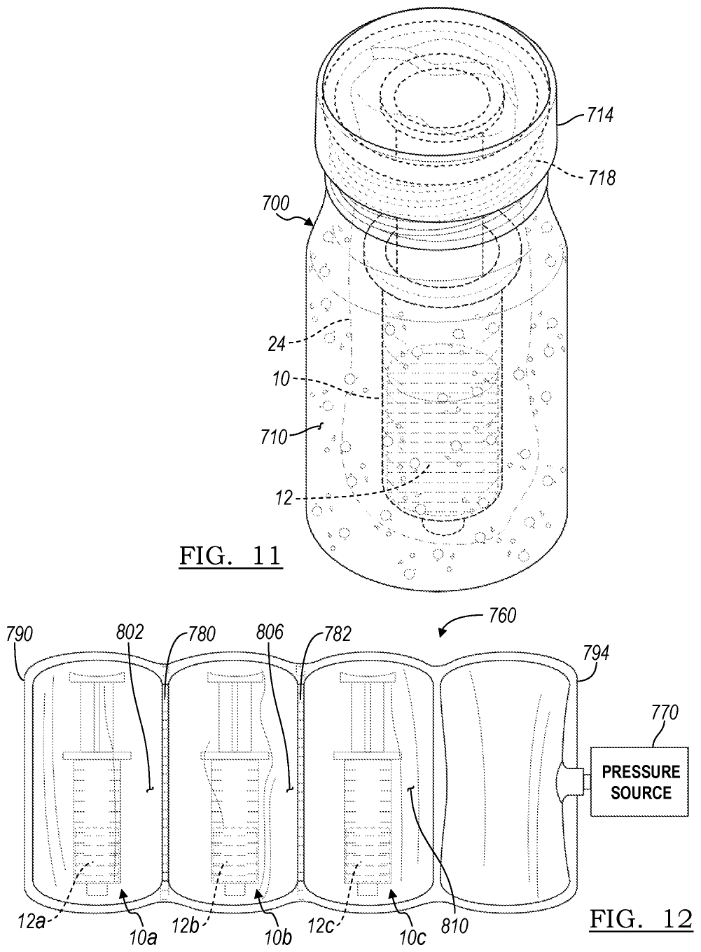

With reference to FIG. 11, a container 700 is illustrated. The container 700 may include an internal volume 710 and a closure or cap 714. The cap 714 may have an engagement connection, such as a threaded engagement 718 with the container 700, similar to the lid 138 discussed above. The internal volume 710 of the container 700, therefore, may be substantially gas and fluid tight. Positioned within the container 700 may be the syringe 10 positioned within the pouch or sleeve 24.

As discussed above in relation to the container 600 and/or 130, the containers may be pressurized with a selected gas. In various embodiments, the container 700, including the internal volume 710 therein, may be pressurized with air or a non-volatile or inert gas, such as nitrogen gas. The container 700 may be pressurized with the nitrogen gas in an appropriate manner such as applying liquid nitrogen or introducing liquid nitrogen into the container 700 (similar to introducing the first form material 620 into the container 600 of FIG. 10A), or pressurizing the container 700 in the appropriate manner as discussed above such as pressurizing the container 130 as illustrated in FIG. 2. Nevertheless, the container 700 may be pressurized with an inert gas that may not be substantially reactive. The inert gas may be different than the gas dissolved within or forming a partial pressure within the fluid 12 within the syringe 10. As discussed above, the gas within the fluid 12 may include oxygen, such as pure oxygen. Accordingly, the container 700 may include a relatively large volume of high pressure air or inert gas that is different than the gas within the syringe 10.

Although the gas within the container 700 may be different than the gas within the fluid 12, the pressure within the container 700 due to the inert gas may maintain the dissolved gas in the liquid 12. The gas in the container 700, such as substantially filling the volume 710 within the container 700, may be a substantially inert gas and/or less expensive than the gas dissolve within the liquid 12 or otherwise differ from the gas within the liquid 12.

Turning reference to FIG. 12, one or more of the syringes, such as a syringe 10a, a syringe 10b, and a syringe 10c may be placed in a selected container or mass sleeve 760. The mass sleeve 760 may be formed of a selected material such as having (i) having multiple layers of material, (ii) formed of as a laminate and/or doped material (e.g. aluminized polymers), (iii) formed as a thick layer (e.g. about 0.05 millimeter (mm) to about 0.07 mm or thicker) of a polymer or other appropriate material. The sleeve 766, regardless of the formation, may be substantially gas impermeable and non-porous.

The large sleeve 766 may be pressurized from a selected pressure source 770 such as with gas to a selected pressure, such as the pressures discussed above. In various embodiments, the respective syringes 10a, 10b, 10c may include respective volumes of fluid 12a, 12b, and 12c that are prefilled and pre-saturated with a selected gas volume and type. Pressurizing the large sleeve 766 with a selected gas, such as the same gas within the syringe 10a, 10b, 10c and/or a different gas (such as an inert gas as discussed in relation to the container 700), may assist in maintaining a selected pressure within the fluid volumes 12a, 12b, and 12c. The large sleeve 766 may then be subdivided in an appropriate manner, such as with ultrasonic and/or heat sealing and/or dividing. Accordingly, each of the syringes 10a, 109b, 10c may be separated by a heat sealed seam 780, 782 as may be ends of the large sleeve 790 and 794. The large sleeve 766 may be sealed at the selected seal points while the pressure from the pressure source 770 is applied to ensure that each of the individual pockets 802, 806 and 810 include a selected pressure from the pressure source 770. Thus, each of the pockets 802, 806, 810 may be filled with a gas (e.g. pure oxygen) at a selected pressure.

Thus, individual packets and/or removable portions of the large sleeve 766 of the syringes may be formed in a substantially non-permeable pouch 766. The individual portions may be formed or cause to be formed by a user separating the portions at the seals, while maintaining a selected pressure within the pouch due to the pressure source 770 delivering a gas to create the pressure to the large sleeve 766 prior to such as immediately prior to and during the heat sealing. Further, individual sleeves, such as the sleeve 24 illustrated in FIG. 1, may be formed of the same or similar non-porous material as discussed above and pressurized as the single sleeve is sealed to have a pressure formed therein.

In various embodiments, after storage of prefilled saline syringes 10a, 10b, 10c, within oxygen gas-filled pockets 802, 806, and 810, each prefilled saline syringes 10a, 10b, 10c can be removed as needed (e.g. separately or individually) from the corresponding pocket, such as with the help of a preformed cut along the outer edge of the pocket. The removed syringe 10 may be used either as a vascular access flush or as an echo contrast agent.

When used for echo contrast agent generation, even at a low oxygen gas partial pressure, for example about 0 psig to about 3 psig, the oxygen gas bubble within the syringe 10 can be agitated effectively throughout the saline by connecting the syringe to an empty sterile syringe via a stopcock and manually flushing between the two syringes as for conventional air/saline echo contrast. Using the flush/agitation approach with the partial pressure of oxygen, for example when compared to agitation of air/saline for contrast generation is the absence and/or reduction of nitrogen gas and the use of sterile oxygen.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. An contrast agent generating system, comprising:
a container having a fluid positioned therein; and
a gas sealing chamber external to the container having a first gas at a selected pressure therein;
wherein the container is selectively positioned within the gas sealing chamber;
wherein the fluid is operable to have a gas dissolved therein.

2. The system of claim 1, wherein the fluid within the container includes a selected volume of the first gas or a second gas dissolved therein.

3. The system of claim 1, wherein the gas sealing chamber is pressurized with the first gas to the selected pressure and the first gas is allowed to diffuse into the container and dissolve in the fluid.

4. The system of claim 1, wherein the container is maintained in the gas sealing chamber until a use of the fluid in the container.

5. The system of claim 1, further comprising:
a turbulence initiator configured to be connected to the container.

6. The system of claim 1, wherein the container is a syringe;
wherein the syringe includes a barrel having a barrel volume with a plunger moveable within the barrel;
wherein the fluid is within the barrel volume within the gas sealing chamber.

7. The system of claim 1, wherein the container is gas permeable.

8. The system of claim 1, wherein the gas sealing chamber is configured to maintain the selected pressure of the gas, wherein the selected pressure of the gas is about 30 psi to about 2000 psi.

9. The system of claim 1, wherein the selected pressure is about 30 psi to about 100 psi.

10. The system of claim 7, wherein the fluid is a low viscosity liquid.

11. The system of claim 8, wherein the low viscosity liquid is a saline solution.

12. The system of claim 9, wherein the saline solution is operable to achieve about 30 psi partial pressure of oxygen gas within the container.

13. The system of claim 1, within the container includes a selected volume of a second gas dissolved therein;
wherein the gas sealing chamber is pressurized with the first gas to the selected pressure and the first gas assists in maintaining a selected pressure external to the container within the gas sealing chamber.

14. The system of claim 13, wherein the first gas is air or an inert gas.

15. The system of claim 13, further comprising:
a sleeve in which the container is placed and both positioned within the gas sealing chamber.

16. The system of claim 1, wherein the gas sealing chamber has the first gas at a selected pressure therein due to a sublimation of a liquid within the gas sealing chamber.

17. A method of generating a contrast agent, comprising:
placing the syringe having a volume of liquid therein in a container;
pressurizing the container with a first gas; and
sealing the container with the syringe placed therein to at least maintain the first gas or a second gas within the liquid within the syringe.

18. The method of claim 17, further comprising:
allowing at least a portion of the first gas to transport into the fluid.

19. The method of claim 17, further comprising:
dissolving a selected partial pressure of the second gas within the liquid prior to placing the syringe within the container; and
maintaining at least a portion of the selected partial pressure of the second gas within the syringe by pressurizing the container with the first gas.

20. The method of claim 17, wherein pressurizing the container with the first gas includes allowing a liquid to sublimate within the container after (i) the container is sealed and (ii) the syringe is placed within the container.

21. A kit for generating a contrast agent comprising:
a first container having a fluid positioned therein positioned within a first gas sealing chamber external to the container; and
at least one connection configured to be connected to the container having a turbulence region;
wherein the gas sealing chamber is pressurized with a gas to a selected pressure and the gas is allowed to dissolve in the fluid.

22. The kit of claim 21, further comprising:
a second container having a fluid positioned therein positioned within a second gas sealing chamber external to the container;
at least one connected configured to be connected to the container having a turbulence region;
wherein the gas sealing chamber is pressurized with a gas to a selected pressure and the gas is allowed to dissolve in the fluid.

23. The kit of claim 21, further comprising:
at least a second container having a fluid positioned therein positioned within a second gas sealing chamber external to the container.

24. An contrast generating system, comprising:
a gas sealing container having:
a volume defining chamber to contain a selected volume of a fluid having a selected gas saturated therein;
a moveable member positioned within the volume defining chamber configured to be moved at a selected time to express the fluid form the volume defining chamber;
wherein the gas sealing container is pressurized with a gas to a selected pressure.

25. The contrast generating system of claim 24, wherein the gas is allowed to dissolve in the fluid over time and be substantially maintained within the volume defining chamber.

26. The contrast generating system of claim 24, wherein the moveable member is a manually operable plunger.
